(19) United States
(12) Patent Application Publication
(10) Pub. No.: US 2025/0040961 A1
(43) Pub. Date: Feb. 6, 2025

(54) STEERABLE ENDOSCOPIC NEEDLE

(71) Applicant: BOSTON SCIENTIFIC MEDICAL DEVICE LIMITED, Galway (IE)

(72) Inventor: Deepak Kumar SHARMA, Muzaffarnagar (IN)

(21) Appl. No.: 18/752,019

(22) Filed: Jun. 24, 2024

Related U.S. Application Data
(60) Provisional application No. 63/517,699, filed on Aug. 4, 2023.

(51) Int. Cl. A61B 17/34; A61B 17/00

(52) U.S. Cl. CPC A61B 17/3478; A61B 2017/00818

(57) ABSTRACT

A device includes a needle, cables and a handle. The needle has a proximal section and a distal section. The distal section is more flexible than the proximal section. The needle also includes channels extending within a wall thereof. Each of the channels extends from a proximal end of the needle to a distal channel end within the distal section. The needle may be inserted into a living body via an insertion instrument. Each of the cables is received within a corresponding one of the channels. A distal end of each of the cables is coupled to the distal channel end of the corresponding channel. During use, the handle remains outside the body. The handle includes an actuator coupled to the cables. Operation of the actuator away from a neutral position places at least one of the cables in tension to curve the distal section of the needle.

### PRIORITY CLAIM

The present disclosure claims priority to U.S. Provisional Patent Application Ser. No. 63/517,699 filed Aug. 4, 2023; the disclosure of which is incorporated herewith by reference.

### FIELD

present disclosure relates to an endoscopic needle and, in particular, an endoscopic ultrasound (EUS) guided access needle.

### BACKGROUND

A hollow needle may be used in an EUS-guided procedure to access target anatomy (e.g., a bile duct), and introduce a guidewire through the needle lumen into the target anatomy to, e.g., guide a stenting procedure, to remove a blockage, etc. In certain procedures, a user may be unable to align an insertion device (e.g., an endoscope) with a desired path of the needle to the target the anatomical structure.

### SUMMARY

The present disclosure relates to a device for treating tissue. The device includes a needle having a proximal section and a distal section. The distal section is more flexible than the proximal section. The needle further includes a plurality of channels extending within a wall thereof. Each of the channels extends from a proximal end of the needle to a distal channel end within the distal section. The needle is configured to be inserted into a living body via an insertion instrument.

The device also includes a plurality of cables. Each of the cables is received within a corresponding one of the channels. A distal end of each of the cables is coupled to the distal channel end of the corresponding channel.

In addition, the device includes a handle. During use, the handle remains outside the body. The handle includes an actuator coupled to the cables. Operation of the actuator away from a neutral position places at least one of the cables in tension to curve the distal section of the needle.

In an embodiment, the needle is hollow and the channels extend through a length of the needle in the wall surrounding a lumen of the needle.

In an embodiment, the channels extend to a distal end of the needle.

In an embodiment, the needle includes a first pair of channels on a first side of the needle and a second pair of channels on a second side of the needle opposite the first side and wherein the device includes a first pair of cables extending through the first pair of channels and a second pair of cables extending through the second pair of channels, proximal ends of the cables of the first and second pairs of cables being connected to the actuator so that, when the actuator is operated in a first direction, the cables of the first pair of cables are tensioned and the cables of the second pair of cables are loosened so that the distal section of the needle is curved toward the first side of the needle.

In an embodiment, the cables are formed of Nitinol and are between 0.1 mm and 0.2 mm in diameter.

In an embodiment, the distal section of the needle includes a plurality of cuts into a wall of the needle, a first portion of the cuts being arranged in a first group extending along the first side of the distal section of the needle.

In an embodiment, a second portion of the cuts are arranged in a second group extending along the second side of the distal section of the needle.

In an embodiment, the cuts of the first group are substantially similar in size and distribution along a length of the distal section of the needle to the cuts of the second group.

In an embodiment, at least one of a spacing and a size of the cuts of the first group varies along a length of the first group.

In an embodiment, at least one of a spacing and a size of the cuts of the second group varies along the length of the second group.

In an embodiment, the needle is configured so that, actuation of the actuator permits bending of the distal section of the needle only in a predefined plane.

In addition, the present disclosure relates to a device for treating tissue. The device includes a needle having a flexible distal section. The needle includes a plurality of channels extending within a wall thereof. Each of the channels extends from a proximal end of the needle to a distal channel end within the distal section. The needle is configured to be inserted into a living body via an insertion instrument.

The device also includes a first cable received within a first one of the channels with a distal end of the first cable coupled to the distal channel end of the first channel. In addition, the device includes a second cable received within a second one of the channels with a distal end of the second cable coupled to the distal channel end of the second channel. The second cable is located on a second side of the needle opposite a first side of the needle on which the first cable is located. The first and second cables extend to a location that remains, during use of the device, outside a patient's body accessible to a user so that the user may tension the first cable to curve the distal section of the needle toward the first side.

In an embodiment, the device further includes a handle which, during use, remains outside the body. The handle includes an actuator coupled to the first and second cables, the actuator being configured so that operation of the actuator away from a neutral position in a first direction placing the first cable in tension to curve the distal section of the needle toward the first side of the needle and operation of the actuator away from a neutral position in a second direction placing the second cable in tension to curve the distal section of the needle toward the second side of the needle.

In addition, the present disclosure relates to a method of treating tissue. The method includes inserting to a target site within a living body an insertion device; inserting through the insertion device a needle having a proximal section and a distal section wherein the distal section is more flexible than the proximal section; extending the distal section of the needle distally out of the insertion device to enter a target anatomical structure; and tensioning at least one cable extending through a wall of the needle to pull the needle into a curved configuration within the target anatomical structure to achieve a desired orientation and positioning of the needle within the target anatomical structure.

In an embodiment, the method further includes operating a handle to orient the needle so that, a plane within which the distal section of the needle curves is in the desired orientation relative to the target anatomical structure before tensioning the at least one cable.

In an embodiment, the target anatomical structure is a bile duct. The method further includes positioning an entirety of the distal section within the bile duct.

In an embodiment, the distal section of the needle includes a plurality of cuts into the wall of the needle, a first portion of the cuts being arranged in a first group extending along a first side of the distal section of the needle, further comprising supplying liquid to a lumen of the needle so that the liquid is supplied to the bile duct via the first group of cuts.

In an embodiment, a second portion of the cuts are arranged in a second group extending along a second side of the distal section of the needle and wherein a plane within which the distal section of the needle curves extends through midpoints of the cuts of the first and second groups of cuts.

In an embodiment, the at least one cable is tensioned by operating an actuator of a handle coupled to the needle.

In an embodiment, the actuator includes a ball joint between two sections of the handle.

### BRIEF DESCRIPTION

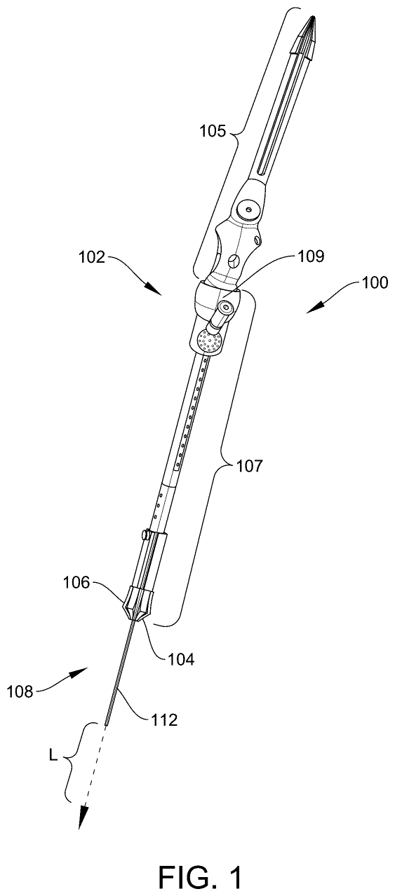

FIG. 1 shows a perspective view of a steerable needle device according to a first embodiment with a needle thereof in a first configuration.

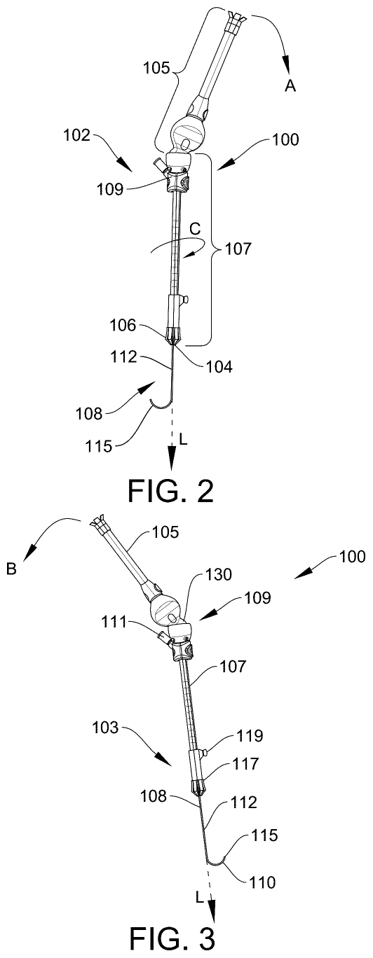

FIG. 2 shows a perspective view of the device of FIG. 1 with a needle extended distally from a distal end of a protective sheath in a second configuration.

FIG. 3 shows a perspective view of the device of FIG. 1 with a needle extended distally from a distal end of a protective sheath in a third configuration.

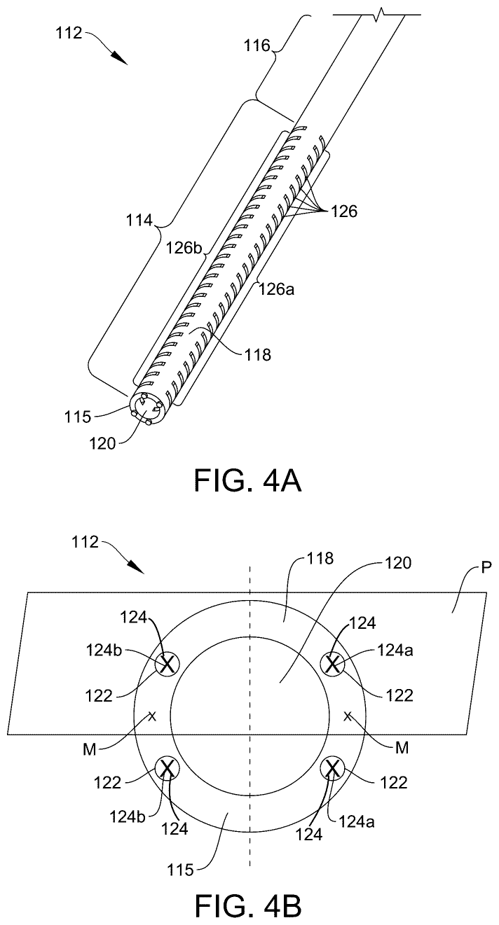

FIG. 4A shows a perspective view of a distal portion of a needle of the device of FIG. 1.

FIG. 4B shows a perspective view of a distal end of the needle of FIG. 4A.

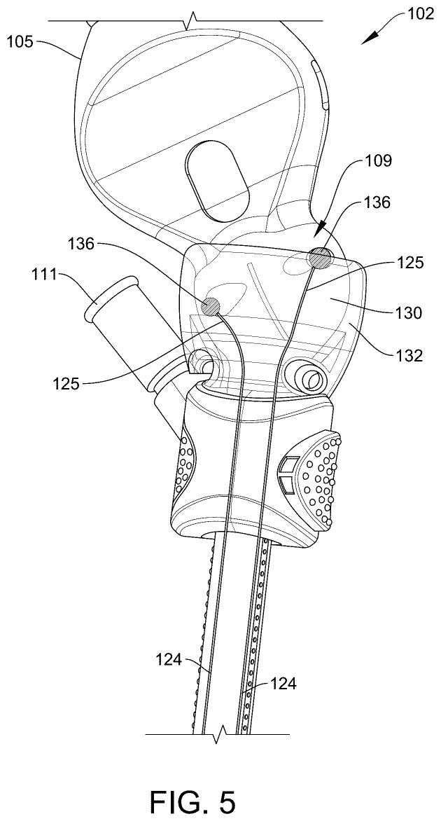

FIG. 5 shows a perspective view of a steering joint of the device of FIG. 1.

### DETAILED DESCRIPTION

The present disclosure may be further understood with reference to the following description and the appended drawings, wherein like elements are referred to with the same reference numerals. The exemplary embodiments describe steerable needle devices that facilitate the insertion of the needles into target anatomical structures.

In this application the terms distal and proximal signify directions away from (distal) and toward (proximal) a user of the device. Thus, for devices according to the described embodiments that are used in conjunction with an insertion device (e.g., an endoscope), the proximal end of the device generally remains outside the body accessible to the user, while the distal end is inserted through the insertion device (e.g., within a working channel of the insertion device) and extended distally out of the distal end of the insertion device into a target anatomical structure.

Those skilled in the art will understand that, although the devices according to the various embodiments are described as used in conjunction with a flexible endoscope to access a bile duct, these devices may be used in conjunction with a wide variety of flexible or rigid insertion devices (e.g., ureteroscopes, lithoscopes, laparoscopes, etc.) to facilitate the insertion of a needle into any desired anatomical structure. As would be understood by those skilled in the art, the needles according to the disclosed embodiments are generally formed of a single material along their lengths (e.g., Nitinol) and include a tissue piercing tip at their distal ends. As would be further understood by those skilled in the art the Nitinol enhances the column strength of the needle making it easier to push the needle through an insertion device even when the insertion device defines a tortuous path. This material may also enhance the transmission of torque along the needle, improve a stiffness of the proximal portions of the needle while allowing the laser cut more flexible distal section to be easily steerable. Thus, a steerable needle as described herein may have a diameter as small as 0.3 mm.

As seen in FIGS. 1-5, a device 100 according to an exemplary embodiment includes a handle 102 extending to a distal end 104 that includes a coupling 106 configured to be coupled to the proximal end of an insertion device. For example, in this embodiment, the coupling 106 is configured to be threaded onto a corresponding coupling at the proximal end of the insertion device (not shown). As will be described in more detail below, the coupling 106 of this embodiment is configured so that the distal end 104 of the handle 102 can rotate relative to the coupling 106 so that the handle 102 can be rotated relative to the coupling 106 without loosening or tightening the connection between the handle 102 and the insertion device. The handle 102 of this embodiment includes a proximal section 105 coupled to a distal section 107 at a joint 109.

The device 100 of this embodiment includes an insertion section 108 that extends distally from the handle 102 and which is configured to be slidably inserted into the insertion device. In this embodiment, as the device 100 may be configured for use in conjunction with a flexible insertion device (e.g., a flexible endoscope), the insertion section 108 is sufficiently flexible to be inserted through the insertion device even when the insertion device has been inserted to a target site within a living body along a tortuous path (e.g., a path defined by a natural body lumen into which the insertion device has been inserted via a natural body orifice) and has a length selected so that, when the coupling 106 is connected to the proximal end of the insertion device, a distal end 110 of the insertion section 108 extends distally out of a distal end of the insertion device by a desired distance (e.g., a distance at least equal to a distance from a distal end of the insertion device when it is in a target position adjacent to a location at which the insertion section 108 is to access the target anatomical structure plus a distance to which the distal end 110 of the insertion section 108 is to be extended into the target anatomical structure.

In addition, the handle 102 includes an insertion port 111 that is in fluid communication with a central lumen 120 of the needle 112 so that fluids introduced into the insertion port 111 are provided to tissue surrounding a distal section 114 of the needle 112. The needle 112 may be formed, for example, from a biocompatible metal such as Nitinol (e.g., a Nitinol hypotube) stainless steel or a plastic such as PEEK or Delrin.

The handle 102 further includes a length adjustment mechanism 103 that includes a slider 117 mounted over the proximal section 105 with a locking nut 119 that can be loosened to permit the slider to slide longitudinally over the proximal section 105 to a desired position so that the length of the needle 112 may be adjusted relative to the length of the insertion device so that a desired amount of the length of the needle 112 is extended distally from the insertion device when the coupling 106 is coupled to the insertion device as desired.

As shown in FIGS. 4A and 4B, a flexible distal section 114 of the needle 112 extends distally from a less flexible proximal section 116 to a distal end 115 of the needle 112. The needle 112 of this embodiment includes an outer wall 118 that surrounds and defines the central lumen 120 that is configured, for example, for the introduction of therapeutic fluids to or the withdrawal of fluids or other substances from the target anatomical structure. A plurality of cable lumens 122 (4 cable lumens 122 in this embodiment) extend through the wall 118 of the needle 112 with each of the cable lumens 122 extending from a proximal end adjacent to the joint 109 to a distal end adjacent to the distal end 115 of the needle 112.

The needle 112 includes a plurality of steering cables 124. Each of the cables 124 fixed to the wall 118 of the needle 112 at the distal end 115 and extending through a corresponding one of the cable lumens 122 to a proximal end 125 that is coupled to the joint 109 as will be described in more detail below. Specifically, the cables 124 are configured so that, when a first pair 124a of the cables 124 is pulled proximally and a second pair 124b of the cables 124 is permitted to extend further distally, the tension applied to one side of the needle 112 bends the needle 112 toward the side of the needle 112 through which the cables 124 of the first pair 124a extend. That is, the distal end 115 of the needle 112 will be deflected away from a longitudinal axis L of the needle 112 so that the distal section 114 of the needle 112 forms an arc which will have a bending radius that becomes progressively smaller as the amount to which the first pair 124a of the cables 124 is drawn proximally increases.

Those skilled in the art will understand that the extent to which the curvature imparted to the needle 112 continues into the proximal section 116 of the needle 112 will depend (as described in more detail below) on the relative flexibility of the proximal and distal sections 116, 114, respectively as well as the length by which the needle 112 extends distally beyond the distal end of the insertion device. Those skilled in the art will understand that the cables 124 may be formed, for example, of Nitinol. This allows for the use of cables 124 that are between 0.1 mm and 0.2 mm in cross-sectional diameter to deliver the pulling forces required to bend the needle 112 over a length of 1 mm to 4.5 mm. This small diameter may be required for example as the wall 118 of the needle 112 may be from 0.01 inches to 0.03 inches in thickness.

As shows in FIG. 4A, the flexibility of the distal section 114 of the needle 112 is enhanced by including therein a plurality of cuts 126 into the wall 118 distributed along the length of the distal section 114. In this embodiment, the cuts 126 are distributed in two separate longitudinal groups. The first group 126a of this embodiment extends along a first side of the needle 112 corresponding to paths of the cable lumens 122 through which the first pair 124a of the cables 124 extend while the second group 126b extends along a second side of the needle 112 (opposite the first side) corresponding to paths of the cable lumens 122 through which the second pair 124b of the cables 124 extend.

The cuts 126 of this embodiment are shown as substantially equally sized and spaced from one another along a length of the distal section 114 with a circumferential length of each of the cuts 126 of the first group 126a being substantially equal to one another as well as being substantially equal to the circumferential length of each of the cuts 126 of the second group 126b. However, those skilled in the art will understand that any or all of the spacing, width (along the axis L) or circumferential length of the cuts 126 may be varied along the length of the distal section 114, may be varied between the first and second groups 126a, 126b and that a length of the distal section 114 may also be varied to achieve any desired bending of the needle 112.

For example, a distal portion of the distal section 114 (e.g., the distal-most 60%-80% of the length of the distal section 114) may be made more flexible than the proximal portion of the distal section 114. As would be understood by those skilled in the art, this may be done by increasing one or both of the width and the circumferential length of the cuts 126, by increasing the number of cuts per unit length or by decreasing a thickness of the wall 118 in the more flexible part of the distal section 114, etc.

Furthermore, as would be understood by those skilled in the art, the distribution of the cuts 126 of this embodiment configures the distal section 114 of the needle 112 to bend in a preselected plane. For example, the distal section 114 of this embodiment is configured to bend (based on manipulation of the cables 124) in a plane P passing through midpoints M between the cable lumens 122 of the first pair 124a of the cables 124 and between the cable lumens 122 of the second pair 124b of the cables 124. Furthermore, those skilled in the art will understand that the cuts 126 will enhance the visibility of the distal section 114 of the needle 112 under ultrasound as the edges of the cuts 126 enhance the reflection of ultrasound energy.

As indicated earlier, the proximal ends 125 of the cables 124 are coupled to the joint 109 so that a user may, by manipulating the proximal section 105 of the handle 102 relative to the distal section 107, apply tension to a desired one of the first pair 124a of the cables 124 or the second pair 124b of the cables 124 to bend the distal section 114 of the needle 112 in a desired direction within the plane P. Specifically, the joint 109 includes a ball 130 formed at a distal end of the proximal section 105 that is rotatably received within a correspondingly shaped cavity 132 formed in the proximal end of the distal section 107.

As would be understood by those skilled in the art, the joint 109 is, in this embodiment, formed so that the ball 130 can rotate within the cavity 132 only in a plane corresponding the distribution of the points at which the cables 124 of the first pair 124a are coupled to the ball 130 and the points at which the cables 124 of the second pair 124b are coupled to the ball 130. More specifically, a proximal portion of each of the cables 124 passes through the ball 130 with a crimp 136 at the proximal end of each of the cables 124 preventing the proximal ends of the cables 124 from being drawn distally through the ball 130. Thus, when the proximal section 105 of the handle 102 is rotated relative to the distal section 107, the cables 124 of one of the first and second pairs 124a, 124b are tensioned and drawn proximally through the needle 112 while the cables 124 of the other pair are correspondingly loosened so that the distal end of the needle 112 is pulled toward the side of the needle 112 within which the tensioned pair of the cables 124 resides.

That is, the joint 109 of this embodiment is formed so that the user may apply tension only to either the first pair 124a or the second pair 124b of the cables 124 and cannot apply tension simultaneously to, for example, a first one of the cables 124 of the first pair 124a and a first one of the cables 124 of the second pair 124b. This ensures that the distal section 114 of the needle 112 is bent only within the plane P. However, as the distal section 107 of the handle 102 is rotatable relative to the coupling 106 (and, consequently to an insertion device coupled thereto), the user may deflect the distal section 114 of the needle 112 in any desired plane by rotating the distal section 107 relative to the insertion device and, consequently, rotating the needle 112 relative to the insertion device. That is, the needle 112 of this embodiment is fixed to the distal section 107 of the handle 102 so that rotating the distal section 107 of the handle 102 rotates the needle 112 and this, in turn, rotates the plane P within the body (i.e., relative to the target anatomical structure).

For example, as shown in FIG. 2, the proximal section 105 is rotated in a first direction A which tensions the first pair 124a of the cables 124 curving the distal end 115 of the needle 112 toward the side of the needle 112 including the first group 126a of cuts 126 while as shown in FIG. 3, the proximal section 105 is rotated in a second direction B which tensions the second pair 124b of the cables 124 curving the distal end 115 of the needle 112 toward the side of the needle 112 including the second group 126b of cuts 126. Those skilled in the art will understand that the further the proximal section 105 is rotated relative to the distal section 107 (e.g., from a neutral position in which a longitudinal axis of the proximal section 105 is aligned with a longitudinal axis of the distal section 107) the more tightly the distal section 114 of the needle 112 will curve (i.e., a bending radius of the curved distal section 114 will decrease).

In use, a user may first adjust the position of the length adjustment mechanism 103 on the proximal section 105 of the handle 102 to an insertion position (e.g., an insertion/retraction configuration) in which the length of the needle 112 is selected so that the distal end 115 of the needle 112 remains within the working channel of the insertion device (e.g., so that the needle 112 does not protrude from the insertion device as the insertion device is inserted to a target location in the body). The user then inserts the needle 112 through the working channel of the insertion device (e.g., the endoscope) and couples the coupling 106 to a corresponding coupling on the proximal end of the insertion device.

The insertion device including the needle 112 housed is then inserted into the body to a target site adjacent (e.g., via a natural body orifice and through a natural body lumen) to a site adjacent to an anatomical structure to be accessed by the needle. The user then readjusts the position of the length adjustment mechanism 103 (e.g., to an operable configuration defined based on a length of the insertion device being used) so that a desired length of the needle 112 (e.g., including at least the distal section 114) sufficient to pass the needle 112 into the target anatomical structure as desired protrudes from the distal end of the insertion device. Those skilled in the art will understand that, if desired, the needle 112 may be inserted into the working channel of the insertion device and coupled thereto after the distal end of the insertion device has been positioned as desired relative to the target anatomical structure.

In this case, the length adjustment mechanism 103 may simply be set to the operable configuration and the coupling 106 then coupled to the insertion device. The user may use a combination of the steering capabilities (if any) of the insertion device and manipulation of the proximal section 105 relative to the distal section 107 of the handle 102 to curve the distal section 114 of the needle 112 to any desired curvature that may be considered helpful in the insertion of the needle 112 into the target anatomical structure. As would be understood by those skilled in the art, the curving of the needle 112 may be helpful if a user loses access to the target anatomical structure during a procedure. For example, if the needle 112 were to slip out of the bile duct, the user may curve the distal section 114 of the needle 112 as desired to facilitate re-insertion of the needle 112 into the bile duct.

The needle 112 may otherwise be inserted into the target anatomical structure in the same manner as would be done with conventional needles. If, for example, the target anatomical structure is a bile duct, the user may insert the insertion device to a target location within the small intestine employing the vision system of the insertion device and additionally using ultrascopic imaging, advance the distal end 115 of the needle 112 distally out of the insertion device until the distal end 115 of the needle 112 (which may include, for example, a tissue piercing tip) penetrates a target site on the wall of the small intestine and then penetrates a wall of the bile duct to enter therein. At this point, using the ultrasonic imaging now that the distal end 115 of the needle 112 is no longer visible using the endoscopic vision system, the user may curve the distal section 114 of the needle 112 as desired by moving the proximal section 105 relative to the distal section 107 in either direction A or B.

For example, if a user wants to point the distal end 115 of the needle 112 upstream within the bile duct, the user will first rotate the handle 102 relative to the insertion device around the longitudinal axis of the distal section 107 in the direction C shown in FIG. 3 until, based on observations of the cuts 126, the user determines that the plane P is aligned to generally include a longitudinal axis of the bile duct. Alternatively, the user may slightly curve the distal section 114 and adjust the rotation of the distal section 107 until the curvature of the distal section 114 generally aligns with the axis of the bile duct. When the user has curved the distal section 114 as desired, the user may further extend the needle 112 distally so that the curved distal section advances into the bile duct in the desired direction. When the needle 112 has been positioned as desired, the user may perform any other desired procedures in the same manner as would be done with a conventional needle.

For example, a user may pass a guidewire through the insertion port 111 and advance the guidewire through the needle into the bile duct so that other devices may be inserted into the bile duct along the guidewire. Furthermore, the length of the first and second groups 126a, 126b may be selected so that, when the needle 112 is positioned as desired, all of the cuts 126 are within the bile duct (or other target anatomical structure). Thus, liquid is provided to the needle 112 via the insertion port 111 will travel through the entire proximal section 116 which has no openings through the wall 118 until it reaches the cuts 126 and flows out into the bile duct. Finally, those skilled in the art will recognize that by controlling an amount of deflection of the proximal section 105 relative to the distal section 107, the user may curve the distal section 114 to any desired amount which allows more control of the degree of curvature than would be afforded, for example, by a needle with a pre-formed J-tip.

Then, when the procedure is complete, the user may either decouple the coupling 106 from the insertion device and withdraw the needle 112 entirely from the insertion device (e.g., leaving the guidewire in place) so that the working channel of the insertion device may then be used for the insertion of other devices into the bile duct over the guidewire. Alternatively, the user may simply adjust the length adjustment mechanism 103 so that the needle 112 is returned to the insertion/retraction configuration in which it is fully housed within the insertion device. The insertion device may then be withdrawn from the body along with the needle 112.

It will be apparent to those skilled in the art that various modifications may be made in the present disclosure, without departing from the scope of the disclosure. Furthermore, those skilled in the art will understand that the features of any of the various embodiments may be combined in any manner that is not inconsistent with the description and/or the functionality of the embodiments. For example, those skilled in the art will understand that any number of cables (2 or more) may be used to achieve the desired curvature of the distal section of the needle. Furthermore, any of a wide variety of known mechanisms may be used to tension one or more cables including on the handle sliders or knobs which, when rotated, wind up or unwind cables and that such mechanisms may be employed in any desired manner to facilitate the curvature of the distal section of a needle by a user.

1-13. (canceled)

14. A device for treating tissue, comprising:
a needle having a proximal section and a distal section wherein the distal section is more flexible than the proximal section, the needle further including a plurality of channels extending within a wall thereof, each of the channels extending from a proximal end of the needle to a distal channel end within the distal section, the needle being configured to be inserted into a living body via an insertion instrument;
a plurality of cables with each of the cables being received within a corresponding one of the channels, a distal end of each of the cables being coupled to the distal channel end of the corresponding channel; and
a handle which, during use, remains outside the body, the handle including an actuator coupled to the cables, operation of the actuator away from a neutral position placing at least one of the cables in tension to curve the distal section of the needle.

15. The device of claim 14, wherein the needle is hollow and the channels extend through a length of the needle in the wall surrounding a lumen of the needle.

16. The device of claim 15, wherein the channels extend to a distal end of the needle.

17. The device of claim 14, wherein the needle includes a first pair of channels on a first side of the needle and a second pair of channels on a second side of the needle opposite the first side and wherein the device includes a first pair of cables extending through the first pair of channels and a second pair of cables extending through the second pair of channels, proximal ends of the cables of the first and second pairs of cables being connected to the actuator so that, when the actuator is operated in a first direction, the cables of the first pair of cables are tensioned and the cables of the second pair of cables are loosened so that the distal section of the needle is curved toward the first side of the needle.

18. The device of claim 14, wherein the cables are formed of Nitinol and are between 0.1 mm and 0.2 mm in diameter.

19. The device of claim 17, wherein the distal section of the needle includes a plurality of cuts into a wall of the needle, a first portion of the cuts being arranged in a first group extending along the first side of the distal section of the needle.

20. The device of claim 19, wherein a second portion of the cuts are arranged in a second group extending along the second side of the distal section of the needle.

21. The device of claim 20, wherein the cuts of the first group are substantially similar in size and distribution along a length of the distal section of the needle to the cuts of the second group.

22. The device of claim 20, wherein at least one of a spacing and a size of the cuts of the first group varies along a length of the first group.

23. The device of claim 22, wherein at least one of a spacing and a size of the cuts of the second group varies along the length of the second group.

24. The device of claim 14, wherein the needle is configured so that, actuation of the actuator permits bending of the distal section of the needle only in a predefined plane.

25. A device for treating tissue, comprising:
a needle having a flexible distal section, the needle including a plurality of channels extending within a wall thereof, each of the channels extending from a proximal end of the needle to a distal channel end within the distal section, the needle being configured to be inserted into a living body via an insertion instrument;
a first cable received within a first one of the channels with a distal end of the first cable coupled to the distal channel end of the first channel; and
a second cable received within a second one of the channels with a distal end of the second cable coupled to the distal channel end of the second channel, the second cable being located on a second side of the needle opposite a first side of the needle on which the first cable is located, the first and second cables extending to a location that remains, during use of the device, outside a patient's body accessible to a user so that the user may tension the first cable to curve the distal section of the needle toward the first side.

26. The device of claim 25, further comprising:
a handle which, during use, remains outside the body, the handle including an actuator coupled to the first and second cables, the actuator being configured so that operation of the actuator away from a neutral position in a first direction placing the first cable in tension to curve the distal section of the needle toward the first side of the needle and operation of the actuator away from a neutral position in a second direction placing the second cable in tension to curve the distal section of the needle toward the second side of the needle.

27. A method for treating tissue, comprising:
inserting to a target site within a living body an insertion device;
inserting through the insertion device a needle having a proximal section and a distal section wherein the distal section is more flexible than the proximal section;
extending the distal section of the needle distally out of the insertion device to enter a target anatomical structure; and
tensioning at least one cable extending through a wall of the needle to pull the needle into a curved configuration within the target anatomical structure to achieve a desired orientation and positioning of the needle within the target anatomical structure.

28. The method of claim 27, further comprising:
operating a handle to orient the needle so that, a plane within which the distal section of the needle curves is in the desired orientation relative to the target anatomical structure before tensioning the at least one cable.

29. The method of claim 27, wherein the target anatomical structure is a bile duct and wherein the method further comprising:
positioning an entirety of the distal section within the bile duct.

30. The method of claim 29, wherein the distal section of the needle includes a plurality of cuts into the wall of the needle, a first portion of the cuts being arranged in a first group extending along a first side of the distal section of the needle, further comprising supplying liquid to a lumen of the needle so that the liquid is supplied to the bile duct via the first group of cuts.

31. The method of claim 30, wherein a second portion of the cuts are arranged in a second group extending along a second side of the distal section of the needle and wherein a plane within which the distal section of the needle curves extends through midpoints of the cuts of the first and second groups of cuts.

32. The method of claim 31, wherein the at least one cable is tensioned by operating an actuator of a handle coupled to the needle.

33. The method of claim 32, wherein the actuator includes a ball joint between two sections of the handle.
